(19) United States
(12) Patent Application Publication
(10) Pub. No.: US 2016/0168615 A1
(43) Pub. Date: Jun. 16, 2016

(54) COMPOSITIONS AND METHODS FOR DETECTING PATHOGENS

(71) Applicant: BugCo, Inc., San Francisco, CA (US)

(72) Inventors: Wendye Robbins, San Francisco, CA (US); Ving J. Lee, Los Altos, CA (US)

(21) Appl. No.: 14/970,286

(22) Filed: Dec. 15, 2015

Related U.S. Application Data
(60) Provisional application No. 62/092,165, filed on Dec. 15, 2014.
(60) Provisional application No. 62/158,761, filed on May 8, 2015.

(51) Int. Cl. C12Q 1/04

(52) U.S. Cl. CPC C12Q 1/04

(57) ABSTRACT

Compositions and methods for detecting pathogens are provided. In one aspect, a method for detecting a pathogen on or within an object comprises: providing a detection agent configured to generate a visible indication when exposed to the pathogen; contacting the detection agent with the object; and visually detecting the presence or absence of the visible indication, wherein the presence of the visible indication indicates that the pathogen is present on or within the object.

### CROSS-REFERENCE

This application claims the benefit of U.S. Provisional Application No. 62/092,165, filed Dec. 15, 2014, and U.S. Provisional Application No. 62/158,761, filed May 8, 2015, the contents of both of which are incorporated by reference herein in their entirety.

### BACKGROUND

Diseases caused by pathogen contamination such as foodborne illnesses and hospital-acquired infections impose a significant burden in terms of mortality, morbidity, and healthcare costs. The incidence of such diseases can potentially be reduced by improved monitoring and detection of pathogen contamination. However, existing approaches for detecting pathogens may require specialized equipment, specialized reagents and prolonged testing times that are not suitable for general use. Moreover, existing detection reagents may be toxic and therefore inappropriate for detecting the presence of and monitoring contamination on living and inert surfaces in food products, medical devices, and other objects that come into contact with the human body.

There is a need to provide improved compositions and methods for detecting pathogens. The present disclosure addresses this need and more.

### SUMMARY

The present disclosure provides compositions and methods for detecting pathogens.

The disclosure provides a method for detecting a pathogen on or within an object, the method comprising: providing a detection agent configured to generate a visible indication when exposed to the pathogen; contacting the detection agent with the object; and visually detecting the presence or absence of the visible indication, wherein the presence of the visible indication indicates that the pathogen is present on or within the object. In certain embodiments, said pathogen is a bacterium, fungus, virus, parasite, prion, or a combination thereof. Pathogens may be selected from Yersinia, Klebsiella, Providencia, Erwinia, Enterobacter, Salmonella, Serratia, Aerobacter, Escherichia, Pseudomonas, Shigella, Vibrio, Aeromonas, Streptococcus, Staphylococcus, Micrococcus, Moraxella, Bacillus, Clostridium, Corynebacterium, Francisella, Haemophilus, Bacteroides, Listeria, Acinetobacter, Brucella, Pasteurella, Flavobacterium, Actionmyces, Nocardia, Campylobacter, Mycobacterium, Candida, adenovirus, arenavirus, coronavirus, rhinovirus, influenza virus, picornavirus, paramyxovirus, reovirus, retrovirus, rhabdovirus, and combinations thereof.

In certain embodiments, said detection agent generates said visible indication when contacted with a plurality of different pathogen types. The detection agent may comprise a colorimetric agent, a fluorescent agent, a luminescent reagent, or a combination thereof. The visible indication may be detectable by an unaided eye. The visible indication may be generated by covalent or non-covalent binding of the detection agent with the pathogen. Said covalent binding may be reversible. In certain embodiments, said visible indication is the emission of light of a first wavelength. Said visible indication may be absent when said detection agent is not bound to said pathogen.

In certain embodiments, said detection agent generates a second visible indication when said detection agent is not exposed to said pathogen. The second visible indication may be the emission of light of a second wavelength. Said first wavelength and said second wavelength may be of different wavelengths. Said first wavelength and said second wavelength may have different emission intensities. In certain embodiments, said emission intensity of said first wavelength is detectably different from said emission intensity of said second wavelength.

In certain embodiments, binding of said detection agent to said pathogen is selective for a component located on said pathogen. The component may be selected from a protein, peptide, lipid, lipopolysaccharide, oligosaccharide, polysaccharide, proteoglycan, glycoprotein, peptidoglycan, or a combination thereof. In certain embodiments, said binding is non-specific binding.

In certain embodiments, said detection agent comprises a specific binding agent selected from DNA, RNA, oligonucleotides, polynucleotides, monoclonal antibodies, polyclonal antibodies, antibody fragments, single chain antibodies, synthetic antibodies, peptides, proteins, receptors, enzymes, DNA aptamers, RNA aptamers, oligosaccharides, lectins, peptoids, zDNA, peptide nucleic acids (PNAs), locked nucleic acids (LNAs), mimetics, small molecular weight compounds, or combinations thereof.

In certain aspects, said visible indication is generated by covalent or non-covalent binding of said detection agent with a component produced by said pathogen. The component may be selected from a protein, peptide, lipid, lipopolysaccharide, oligosaccharide, polysaccharide, proteoglycan, glycoprotein, peptidoglycan, and combinations thereof. The component may be selected from a metabolic byproduct of said pathogen. Said metabolic byproduct is selected from carbon dioxide, ammonium, ammonia, hydrogen sulfide, sulfur dioxide, hydrogen, lactate, lactic acid, carbonic acid, sulfuric acid, and combinations thereof. In certain embodiments, said component is a gene product of said pathogen, such as a gene product selected from beta-lactamase, LpxA, LpxB, LpxC, LpxD, and combinations thereof.

In certain embodiments. said detection agent comprises a chromogenic substrate that generates a third visible indication upon binding with said component. In certain embodiments, said component oxidizes or reduces said chromogenic substrate. The component may be an enzyme and said substrate may be a substrate for said enzyme. The chromogenic substrate may be a chromogenic cephalosporin.

The detection agent may be provided in a solution. In certain embodiments, contacting the detection agent with the object comprises applying the solution to one or more portions of the object. The solution may further comprise an additional detection agent configured to provide a fourth visible indication when exposed to a second pathogen. The solution may comprise an environmentally acceptable solvent, such as water, ethanol, polyethylene glycol, propylene glycol and combinations thereof. Applying the solution to said one or more portions of said object may comprise spraying, flowing, dripping, or wiping the solution onto said one or more portions.

Applying the solution to said one or more portions of said object may comprise immersing said one or more portions into said solution.

In certain embodiments, said detection agent is embedded within said object. In certain embodiments, any one or more of said visible indication, said second visible indication, said third visible indication and said fourth visible indication is visually detectable from said exterior of the object. The object may comprise a resin, wherein said detection agent is embedded in said resin. The detection agent may be associated with a film, coating, or layer located on a surface of said object. The detection agent may be embedded within said film, coating, or layer located on said surface of said object.

The detection agent may be generally non-toxic. The detection agent may be generally non-staining. In certain embodiments, said object is located in a food preparation setting, a healthcare setting, or a laboratory setting. One or more of said visible indication, said second visible indication, said third visible indication, and said forth visible indication may be a temporary visual indication. Said temporary visual indication may be unstable when exposed to light. Detection agents may be selected from alizarin, alizarin yellow, basonyl green, basonyl blue, bromochlorophenol blue, bromoxylenol blue, bromophenol blue, bromopyrogallol red, bromothymol blue, bromocresol green, bromocresol purple, chlorophenol red, congo red, o-cresolphthalein, m-cresol purple, cresol red, m-cresol red, crystal violet, erichrome blue black R, erthyrosine powder, ethyl orange, Evans blue, fast sulphon black F, FD&C #2 indigotene, FD&C #2 lake, FD&C #1 triphenylmethane, FD&C #1 lake, FD&C #5 yellow, pyrazoine, FD&C #3 green, FD&C #3 red, FD&C #5 yellow lake, hydroxy naphthol blue, litmus powder, malachite green, malachite green oxalate, methyl orange, methyl red, methyl yellow, methylthymol blue, methyl violet, murexide powder, p-naphtholbenzein, neutral red, nitrazine yellow, pentamethoxy red, phenol red, phenophthalein, rhodamine 6 G, thymol blue, thymophthalein, triarylmethane (pylam blue), xylenol blue, and combinations thereof.

Detection agents may be conjugated to an antibiotic or a non-antibacterial analog thereof. For example, the detection agent may be conjugated to a beta-lactam antibiotic or a non-antibacterial analog thereof.

In certain aspects, said detection agent comprises at least one chromogenic agent and a substrate, wherein said substrate reacts with a component produced by the pathogen thereby generating a reaction product, and said reaction product reacts with said chromogenic agent to generate a fifth visible indication. In certain embodiments, said substrate comprises a beta-lactamase inhibitor and said component comprises a beta-lactamase. Said beta-lactamase inhibitor may comprise a penicillanic acid sulfone or a cephem acid sulfone. Said chromogenic agent may comprise a dye. Dyes may be selected from a triphenylene class dye, an azo class dye, an indigo class dye, and combinations thereof. Cationic triphenylene class dyes may be selected from fuschin, malachite green, malachite green oxalate, thymol blue, crystal violet, and combination thereof. Azo class dyes may be selected from methyl yellow, methyl orange, and combinations thereof. Indigo class dyes may be selected from indigo carmine, indigo, and combinations thereof. In certain embodiments, said fifth visible indication comprises a color change in said chromogenic agent.

In one aspect of the present disclosure, a method for detecting a pathogen on or within an object is provided. The method comprises: providing a detection agent configured to generate a visible indication when exposed to the pathogen; contacting the detection agent with the object; and visually detecting the presence or absence of the visible indication, wherein the presence of the visible indication indicates that the pathogen is present on or within the object.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A and 1B illustrate detection agents producing a visible indication when exposed to a pathogen.

FIGS. 2A through 2C illustrate a detection agent providing a temporary visible indication when exposed to a pathogen.

FIG. 3 illustrates a method for detecting a pathogen on or within an object.

FIG. 4 illustrates detection of bacteria using a beta-lactamase inhibitor and methylene blue dye.

### DETAILED DESCRIPTION

The present disclosure relates generally to compositions and methods for detecting pathogens such as bacteria, fungi, and viruses. In certain aspects, the present disclosure describes detection agents that provide a visible indication (e.g., visible to the naked or unaided eye) when exposed to one or more pathogens and/or one or more pathogen types. In certain aspects, the detection agent is contacted with an object (e.g., an object in a food preparation setting, healthcare setting, laboratory setting, etc.) and the presence or absence of the visible indication is visually detected in order to determine whether a pathogen is present on or within the object. In certain aspects, the presence of the visible indication indicates that the pathogen is present on or within the object. Various aspects of the present disclosure provide rapid pathogen detection without requiring specialized equipment and toxic reagents, thereby enhancing versatility and convenience of assessing and monitoring pathogen contamination for diverse object types. Moreover, some aspects presented herein provide detection agents that produce a temporary visible indication that rapidly appears and disappears after a certain length of time, thereby allowing for pathogen detection without staining the tested object.

Various aspects of the present disclosure are applicable to the detection of a wide variety of pathogen types. In certain aspects, a pathogen encompasses any entity or agent capable of causing disease in humans, animals, plants, or combinations thereof. The pathogens described herein include living entities (e.g., prokaryotic organisms, eukaryotic organisms) and non-living entities (e.g., infectious particles, infectious proteins). Exemplary pathogen types suitable for use with the aspects provided herein include but are not limited to: bacteria, fungi, viruses, parasites, prions, or combinations thereof. Examples of bacteria include but are not limited to the following genera: Yersinia, Klebsiella, Providencia, Erwinia, Enterobacter, Salmonella, Serratia, Aerobacter, Escherichia, Pseudomonas, Shigella, Vibrio, Aeromonas, Streptococcus, Staphylococcus, Micrococcus, Moraxella, Bacillus, Clostridium, Corynebacterium, Francisella, Haemophilus, Bacteroides, Listeria, Acinetobacter, Brucella, Pasteurella, Flavobacterium, Actinomyces, Nocardia, Mycobacterium, or Campylobacter. Examples of fungi include but are not limited to the following genera: Candida, Blastomyces, Aspergillus, Coccidioides, Cryptococcus, Histoplasma, Pneumocystis, or Stachybotrys. Examples of viruses include but are not limited to the following: adenovirus, arenavirus, coronavirus, rhinovirus, influenza virus, picornavirus, paramyxovirus, reovirus, retrovirus, or rhabdovirus.

Certain aspects of the present disclosure provide detection agents that are adapted to detect any desired number or combination of pathogens. In some aspects, a detection agent is adapted to detect a single pathogen type. In alternative aspects, the detection agent is adapted to detect multiple pathogen types, such as two, three, four, five, six, seven, eight, nine, ten or more unique different pathogen types. In certain aspects, the detection agent is adapted to detect 20, 30, 40, 50, 60, 70, 80, 90, 100, 500, or 1000 different pathogen types. In certain aspects, the detection agents described herein are capable of detecting a plurality of related pathogen types, e.g., Gram-positive bacteria or Gram-negative bacteria. For example, in some aspects, the detection agent detects only Gram-positive bacteria. In alternative aspects, the detection agent detects only Gram-negative bacteria. In other aspects, the detection agent detects both Gram-positive and Gram-negative bacteria.

In some aspects of the present disclosure, the detection agent is capable of distinguishing between live pathogens and dead pathogens. For example, in certain aspects, the detection agent detects only live pathogens. In other aspects, the detection agent detects only dead pathogens. In alternative aspects, the detection agent does not distinguish between live and dead pathogens and is capable of detecting both.

In some aspects, a detection agent detects a pathogen by detecting one or more components or products associated with the pathogen. Examples of such components or products include but are not limited to: proteins, peptides, lipids, lipopolysaccharides, oligosaccharides, polysaccharides, proteoglycans, glycoproteins, peptidoglycans, nucleic acids, small molecules, ions, or combinations thereof. In some aspects, the components or products form part of the pathogen, e.g., are located on a surface of the pathogen or within the pathogen. Examples of such components or products include but are not limited to: cell wall components (e.g., peptidoglycans, techoic acids), cell membrane components (e.g., transmembrane channels or transporters, adhesion proteins, receptors, surface proteins, phospholipids, lipopolysaccharides), extracellular components (e.g., fimbrae, pili, flagella, glycocalyx), intracellular components (e.g., organelles, cytoskeleton components, vesicles, vacuoles), viral structural components (e.g., capsid proteins, coat proteins, envelope components), or combinations thereof. In some aspects, the components or products do not form part of the pathogen, such as: secreted components or products; products, byproducts, intermediates, or catalysts for biological processes; or combinations thereof. Examples of such components or products include but are not limited to: metabolic byproducts (e.g., carbon dioxide, ammonium, ammonia, hydrogen sulfide, sulfur dioxide, hydrogen, lactate, lactic acid, carbonic acid, sulfuric acid), signaling molecules (e.g., quorum sensing molecules), extracellular matrix components (e.g., exopolysaccharides), gene products (e.g., enzymes such as beta-lactamase, LpxA, LpxB, LpxC, LpxD), or combinations thereof. In various aspects of the present disclosure, the detection agent is configured to detect a beta-lactamase secreted by a pathogen, such as a class A beta-lactamase, class B beta-lactamase, class C beta-lactamase, class D beta-lactamase, or combinations thereof.

In some aspects, the detection agents described herein enable pathogen detection by providing a visible indication in order to indicate the presence of a pathogen. In some aspects, the visible indication involves a change in the optical characteristics (e.g., color, fluorescence, and/or luminescence) of the detection agent, such that the detection agent exhibits a first set of optical characteristics in the absence of a pathogen and a second, different set of optical characteristics in the presence of a pathogen. Examples of such changes include but are not limited to: the appearance of color, fluorescence, and/or luminescence; the disappearance of color, fluorescence, and/or luminescence; a change in the wavelength of the color, fluorescence, and/or luminescence; a change in the intensity of the color, fluorescence, and/or luminescence; or combinations thereof. In certain embodiments, the visible indication is the emission of light of a first wavelength. In certain embodiments, the first wavelength is selected from a wavelength in the visible spectrum. In certain embodiments, the visual indication is generated by covalent or non-covalent binding of the detection agent with the pathogen.

In certain embodiments, the detection agent generates a second visible indication when said detection agent is not exposed to said pathogen. The second visible indication may be the emission of light of a second wavelength. In certain embodiments, the visible indication and second indication are different, for example the first wavelength and second wavelengths are different, e.g. the visible indication is red and the second visible indication is blue. In certain embodiments, the difference in the first wavelength and second wavelength is visible to the unaided eye such that one can visually determine the presence of absence of the pathogen by looking at the object. In certain embodiments, the visible indication and second visible indication have different emission intensities. In certain embodiments, the emission intensity of the first wavelength is detectably different, e.g., by the unaided eye, than the emission intensity of said second wavelength. In certain embodiments, the first wavelength and the second wavelength are the same or substantially similar but the emission intensity of the visible indication and second visible indication is different such that the difference is detectable, e.g., detectable by the unaided eye.

In certain embodiments, a visible indication described herein may be referred to as a visible indication, a second visible indication, a third visible indication, a forth visible indication or a fifth visible indication. Each of the visible indications described herein may be individually selected from any visible wavelength and emission intensity. In certain embodiments, wherein the detection agent has more than one visible indications, e.g., a visible indication and a second visible indication, the two or more visible indications may emit light of the same or substantially similar wavelengths, the same or substantially similar emission intensities, visibly different light wavelengths, visibly different emission intensities or any combination thereof. In certain embodiments, any visible indication described herein may be visually detectable from said exterior of the object. In certain embodiments, any visual indication described herein may be a temporary visible indication.

In certain aspects, the visible indication is irreversible, in that the detection agent does not revert back to its original optical characteristics once a change has occurred. In certain embodiments, the binding of the pathogen to the detection agent is covalent. In alternative aspects, the visible indication is reversible, in that the detection agent is capable of reverting back to its original optical characteristics after a change has occurred. In certain embodiments, the binding of the pathogen to the detection agent is non-covalent. An example of an irreversible visible indication is a detection agent that is initially uncolored in the absence of pathogen, changes to a predetermined color in the presence of a pathogen, and remains colored when the pathogen is removed. An example of a reversible visible indication is a detection agent that is initially uncolored in the absence of pathogens, changes to a predetermined color when a pathogen is present, and reverts to being uncolored when the pathogen is removed.

In some aspects, any visible indication described here is visible to the naked or unaided human eye, e.g., visually detectable without the use of visualization and/or detection devices such as magnifying glasses or lenses, microscopes, plate readers, spectrophotometer, fluorimeters, luminometers, flow cytometers, and the like. In certain aspects, any visible indication described herein is visually detectable (e.g., with the naked or unaided human eye) without the use of an excitation light source, such as lasers, light-emitting diodes (LEDs), or lamps. In alternative aspects, the visible indication and/or second visible indication is visually detectable (e.g., with the naked or unaided human eye) with the use of an excitation light source.

A wide variety of detection agents are suitable for use with the various aspects of the present disclosure Exemplary types of detection agents include but are not limited to: small molecules, peptides, proteins, lipids, sugars, nucleic acids, polymers, dendrimers, particles, beads, or combinations thereof. In some aspects, the detection agents include a visual indicator (e.g., a colorimetric agent, fluorescent agent, a luminescent agent, or a combination thereof) adapted to produce the visible indication. In some aspects, the visual indicator is a chromogenic substance having one or more chromophores, a fluorogenic substance having one or more fluorophores, and/or a luminogenic substance having one or more luminophores. Exemplary visible indicators include but are not limited to: alizarin, alizarin yellow, basonyl green, basonyl blue, bromochlorophenol blue, bromoxylenol blue, bromophenol blue, bromopyrogallol red, bromothymol blue, bromocresol green, bromocresol purple, chlorophenol red, congo red, o-cresolphthalein, m-cresol purple, cresol red, m-cresol red, crystal violet, erichrome blue black R, erthyrosine powder, ethyl orange, Evans blue, fast sulphon black F, FD&C #2 indigotene, FD&C #2 lake, FD&C #1 triphenylmethane, FD&C #1 lake, FD&C #5 yellow, pyrazoine, FD&C #3 green, FD&C #3 red, FD&C #5 yellow lake, hydroxy naphthol blue, litmus powder, malachite green oxalate, methyl orange, methyl red, methyl yellow, methylthymol blue, methyl violet, murexide powder, p-naphtholbenzein, neutral red, nitrazine yellow, pentamethoxy red, phenol red, phenophthalein, thymol blue, thymophthalein, triarylmethane (pylam blue), xylenol blue, or a combination thereof.

In some aspects, the visual indicator is a dye or a dye derivative that is adapted to provide any one of the visible indications. Exemplary dyes suitable for use with various aspects of the present disclosure include anionic dyes, cationic dyes, and non-ionic dyes. Direct dyes, acidic dyes, and reactive dyes are examples of anionic dyes. Basic dyes and disperse dyes are examples of cationic and non-ionic dyes. In certain aspects, the dye is an azo dye or anthraquinone dye that is modified with various types of functional groups, e.g., vinyl sulfone, chlorotriazine, trichloropyrimidine, difluorochloropyrimidine, or combinations thereof. In certain aspects, the dye is malachite green, rhodamine 6 G, evans blue, or a combination thereof, or a derivative thereof.

The properties of the dyes provided herein can be varied as desired. In certain aspects, the dye is relatively stable, e.g., to light, heat, oxidizing agents, or combinations thereof. In certain aspects, the dye is a biologically degradable dye. In other aspects, the dye is a non-degradable dye. In some aspects, the dye is adapted for rapid bio sorption onto a pathogen (e.g., onto an external surface of a pathogen such as bacterial cell wall). The biosorption kinetics can be influenced by various factors, including but not limited to: local concentration, temperature, bioabsorbant uptake capacity, surface binding, metabolism, time, or combinations thereof. In some aspects, the structure and/or composition of the dye is modified in order to achieve desired dye properties, e.g., stability, degradability, biosorption kinetics, uptake kinetics, nontoxicity. For instance, the dye can be modified in order to provide a temporary visible indication, as described further herein. Exemplary techniques that can be used to modify a dye include but are not limited to: introduction of metabolic breaking points, addition of permeability enhancers (e.g., efflux pump inhibitors, non-specific enhancers such as polyethylene glycol, polycationic agents), ionic strength adjustments, addition of a metallic center, or combinations thereof.

In some aspects of the present disclosure, the detection agent produces the visible indication when exposed to one or more pathogen types. In certain aspects, exposure of a detection agent to a pathogen involves contacting the detection agent with the pathogen and/or contacting the detection agent to a component or product of the pathogen, e.g., any combination of the components or products described in the present disclosure. In certain aspects, exposure of a detection agent to a pathogen involves contacting the detection agent with the component or product without contacting the pathogen itself. In certain aspects, exposure of a detection agent to a pathogen involves interaction of the detection agent with the pathogen and/or interaction of the detection agent with a component or product of the pathogen. In certain aspects, exposure of a detection agent to a pathogen involves interaction of the detection agent with the component or product without interaction with the pathogen.

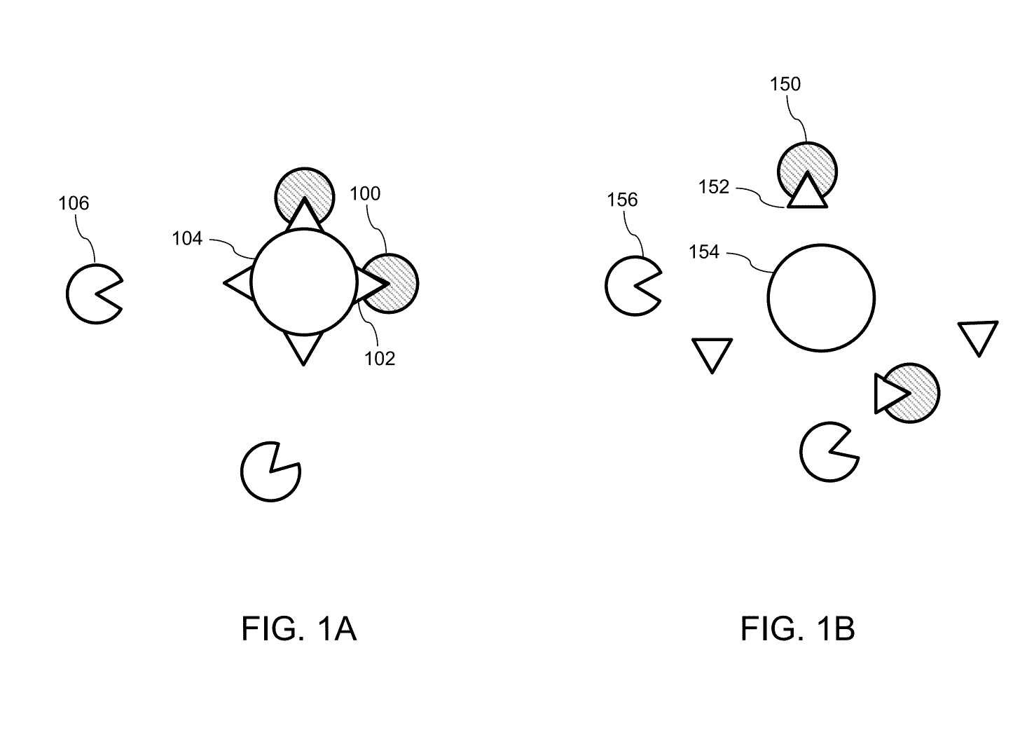

FIGS. 1A and 1B illustrate detection agents producing a visible indication when exposed to pathogen, in accordance with an aspect of the present disclosure. FIG. 1A illustrates detection agents 100 producing a visible indication (represented by shading) upon interaction with components 102 located on the external surface of a pathogen 104. Detection agents 106 that are not interacting with the pathogen components do not produce any visible indication. FIG. 1B illustrates detection agents 150 producing a visible indication (represented by shading) upon interaction with components 152 secreted by a pathogen 154. Detection agents 156 that are not interacting with the secreted components do not produce any visible indication.

Various aspects of the present disclosure enable rapid production of any one of the visible indications described herein following exposure of the detection agent to a pathogen. In some aspects, the visible indication is produced instantaneously or approximately instantaneously when the detection agent is exposed to the pathogen. In some aspects, the visible indication is produced no more than about 1 ms, 10 ms, 50 ms, 100 ms, 200 ms, 500 ms, 1 s, 2 s, 3 s, 5 s, 10 s, 30 s, 45 s, 1 minute, 2 minutes, 5 minutes, 10 minutes, 20 minutes, 30 minutes, 40 minutes, 50 minutes, 1 hour, 2 hours, or 5 hours following exposure of the detection agent to the pathogen. In some aspects, the visible indication is visually detectable (e.g., with the naked or unaided human eye) instantaneously or approximately instantaneously when the detection agent is exposed to the pathogen. In some aspects, the visible indication is visually detectable no more than about 1 ms, 10 ms, 50 ms, 100 ms, 200 ms, 500 ms, 1 s, 2 s, 3 s, 5 s, 10 s, 30 s, 45 s, 1 minute, 2 minutes, 5 minutes, 10 minutes, 20 minutes, 30 minutes, 40 minutes, 50 minutes, 1 hour, 2 hours, or 5 hours following exposure of the detection agent to the pathogen.

In certain aspects, the detection agent provides a temporary visible indication that is only visually detectable for a certain length of time. For example, in some aspects, the temporary visible indication is visually detectable for no more than about 500 ms, 1 s, 2 s, 3 s, 5 s, 10 s, 30 s, 45 s, 1 minute, 2 minutes, 5 minutes, 10 minutes, 20 minutes, 30 minutes, 40 minutes, 50 minutes, 1 hour, 2 hours, or 5 hours. In various aspects, the temporary visible indication appears (e.g., becomes visually detectable) after a certain time following exposure to the pathogen, then disappears (e.g., becomes visually undetectable) after a certain time following exposure to the pathogen. For example, in some aspects, the temporary visible indication appears after no more than about 1 ms, 10 ms, 50 ms, 100 ms, 200 ms, 500 ms, 1 s, 2 s, 3 s, 5 s, 10 s, 30 s, 45 s, 1 minute, 2 minutes, 5 minutes, 10 minutes, 20 minutes, 30 minutes, 40 minutes, 50 minutes, 1 hour, 2 hours, or 5 hours following exposure of the detection agent to the pathogen. In some aspects, the temporary visible indication disappears after no more than about 1 ms, 10 ms, 50 ms, 100 ms, 200 ms, 500 ms, 1 s, 2 s, 3 s, 5 s, 10 s, 30 s, 45 s, 1 minute, 2 minutes, 5 minutes, 10 minutes, 20 minutes, 30 minutes, 40 minutes, 50 minutes, 1 hour, 2 hours, or 5 hours following exposure of the detection agent to the pathogen.

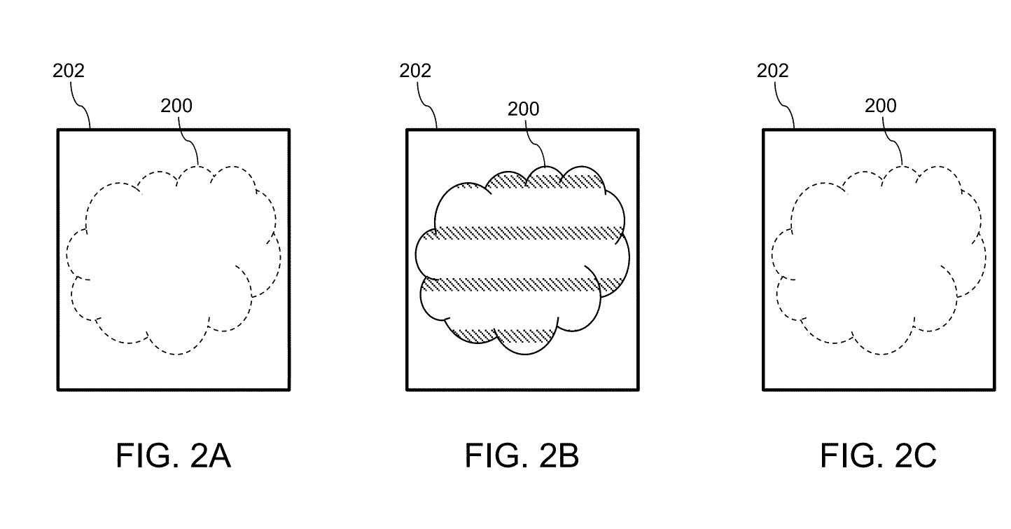

FIGS. 2A through 2C illustrate a detection agent 200 providing a temporary visible indication when exposed to a pathogen, in accordance with an aspect of the present disclosure. In FIGS. 2A through 2C, the detection agent 200 is applied to an object 202 contaminated with a pathogen. FIG. 2A illustrates the detection agent 200 and object 202 at a first time point in which the temporary visible indication has not yet appeared (e.g., the detection agent 200 is initially uncolored). FIG. 2B illustrates the detection agent 200 and object 202 at a second, subsequent time point in which the temporary visible indication has appeared (e.g., the detection agent 200 exhibits visually detectable color, indicated by shading). FIG. 2C illustrates the detection agent 200 and object 202 a third, subsequent time point in which the temporary visible indication has disappeared (e.g., the detection agent 200 is again uncolored).

Various mechanisms can be used to produce a detection agent that provides a temporary visible indication. In some aspects, the detection agent includes a temporary visual indicator (e.g., a dye) that degrades, is consumed, and/or is unstable (e.g., over time; when exposed to light; due to flocculation, flotation, precipitation, oxidation, reduction, ozonization, coagulation, membrane separation, absorption, or combinations thereof) in order to produce the temporary visible indication. Exemplary temporary visual indicators include but are not limited to the following compositions and/or derivatives thereof: alizarin, alizarin yellow, basonyl green, basonyl blue, bromochlorophenol blue, bromoxylenol blue, bromophenol blue, bromopyrogallol red, bromothymol blue, bromocresol green, bromocresol purple, chlorophenol red, congo red, o-cresolphthalein, m-cresol purple, cresol red, m-cresol red, crystal violet, erichrome blue black R, erthyrosine powder, ethyl orange, Evans blue, fast sulphon black F, FD&C #2 indigotene, FD&C #2 lake, FD&C #1 triphenylmethane, FD&C #1 lake, FD&C #5 yellow, pyrazoine, FD&C #3 green, FD&C #3 red, FD&C #5 yellow lake, hydroxy naphthol blue, litmus powder, malachite green, malachite green oxalate, methyl orange, methyl red, methyl yellow, methylthymol blue, methyl violet, murexide powder, p-naphtholbenzein, neutral red, nitrazine yellow, pentamethoxy red, phenol red, phenophthalein, rhodamine 6 G, thymol blue, thymophthalein, triarylmethane (pylam blue), xylenol blue, or a combination thereof.

In various aspects of the present disclosure, the visible indication is generated by interaction of the detection agent with the pathogen and/or interaction of the detection agent with a component or product of the pathogen. Examples of interactions include but are not limited to binding interactions with the detection agent, uptake of the detection agent, reaction with the detection agent, or suitable combinations thereof. A binding interaction can be a reversible or irreversible binding interaction. The binding interactions described herein include nonspecific binding interactions (e.g., due to electrostatic interactions, hydrophobic interactions, hydrophilic interactions, or combinations thereof) as well as specific binding interactions in which the detection agent recognizes and binds to a target (or is recognized by and bound by a target). The pathogen components or products previously described herein are examples of binding targets for a detection agent. For example, in certain aspects, the specific binding target of a detection agent is a component located on a surface (e.g., external surface) of the pathogen. In some aspects, the specific binding target is a component of a cell wall of a bacterium (e.g., a Gram-positive bacterium or a Gram-negative bacterium). As another example, in certain aspects, the specific binding target is a component located internally within the pathogen (e.g., an intracellular component). In yet another example, in certain aspects, the specific binding target is a component or product produced (e.g., secreted) by the pathogen.

In some aspects, a detection agent adapted for specific binding to a pathogen target includes a specific binding agent adapted to recognize and bind to the target. Examples of specific binding agents include but are not limited to: DNA, RNA, oligonucleotides, polynucleotides, monoclonal antibodies, polyclonal antibodies, antibody fragments, single chain antibodies, synthetic antibodies, peptides, proteins, receptors, enzymes, DNA aptamers, RNA aptamers, oligosaccharides, lectins, synthetic nucleic acids, mimetics, small molecular weight compounds, or combinations thereof. In some aspects, the specific binding agent recognizes a single binding target. In alternative aspects, the specific binding agent recognizes a plurality of different target types, e.g., binding targets with related structure and/or compositions.

In various aspects, the detection agent produces a visible indication when bound to a pathogen, and/or to a component or product thereof. For example, in some aspects, the visible indication is caused by one or more optical characteristics of the detection agent being detectably different (e.g., visually detectable to the naked or unaided human eye) when the detection agent is bound versus when the detection agent is unbound. In certain aspects, the altered optical characteristics include one or more spectral properties of the detection agent (e.g., excitation wavelength, emission wavelength, emission intensity). The present disclosure contemplates both reversible and irreversible changes in the optical characteristics of the detection agent. For example, in certain aspects, the binding interaction is reversible and the change in optical characteristics is reversible, such that the detection agent is adapted to have the same optical characteristics (e.g., spectral properties) after being dissociated (e.g., from the pathogen and/or from a component or product thereof) as before binding. In alternative aspects, the binding interaction is reversible but the change in optical characteristics is irreversible, such that the optical characteristics do not revert to the original unbound state even if the detection agent is subsequently dissociated.

In certain aspects, the detection agent includes a substrate that, upon interaction with the pathogen and/or a component or product thereof, forms a product that provides the visible indication. Examples of such substrates include but are not limited to: chromogenic substrates that form a colored product, fluorogenic substrates that form a fluorescent product, luminogenic substrates that form a luminescent product, or combinations thereof. In some aspects, the optical characteristics (e.g., spectral properties) of the product are detectably different from the optical characteristics of the substrate so as to provide the visible indication.

The present disclosure contemplates various mechanisms by which the pathogen or a component or product thereof interact with the detection agent substrate in order to produce the visible indication. In certain aspects, the substrate reacts with a component or product of the pathogen. Examples of such components or products include but are not limited to: cell wall components (e.g., peptidoglycans, techoic acids), cell membrane components (e.g., transmembrane channels or transporters, adhesion proteins, receptors, surface proteins, phospholipids, lipopolysaccharides), extracellular components (e.g., fimbrae, pili, flagella, glycocalyx), intracellular components (e.g., organelles, cytoskeleton components, vesicles, vacuoles), viral structural components (e.g., capsid proteins, coat proteins, envelope components), or combinations thereof. In some aspects, the components or products do not form part of the pathogen, such as: secreted components or products; products, byproducts, intermediates, or catalysts for biological processes; or combinations thereof. Examples of such components or products include but are not limited to: metabolic byproducts (e.g., carbon dioxide, ammonium, ammonia, hydrogen sulfide, sulfur dioxide, hydrogen, lactate, lactic acid, carbonic acid, sulfuric acid), signaling molecules (e.g., quorum sensing molecules), extracellular matrix components (e.g., exopolysaccharides), gene products (e.g., enzymes such as beta-lactamases, LpxA, LpxB, LpxC, LpxD), or combinations thereof. Examples of reactions with a component or product of the pathogen include but are not limited to: redox reactions, acid-base reactions, precipitation reactions, complexation reactions, enzymatic reactions, or combinations thereof. For example, in some aspects, the detection agent includes a chromogenic substrate that is oxidized or reduced by a component or product of the pathogen (e.g., a metabolic byproduct) to form a colored product. As another example, in some aspects, the detection agent includes a chromogenic substrate that is a substrate for an enzyme produced by the pathogen. In certain aspects, the chromogenic substrate is a chromogenic cephalosporin (e.g., nitrocefin, PADAC) that is a substrate for beta-lactamase enzymes produced by the pathogen. The reaction between the substrate and pathogenic component or product can involve binding of the substrate to the component or product, e.g., binding mediated by one or more of the specific binding agents provided herein.

In some aspects, the detection agent includes a substrate including a releasable chromogenic agent, and interaction of the substrate with a pathogenic component or product triggers the release of the chromogenic agent. In certain aspects, the chromogenic agent is “silent” prior to release, and provides the visible indication (e.g., visible color) only when released from the substrate. For example, in some aspects, the substrate includes a modified cephalosporin that releases a chromogenic agent upon reaction with a beta-lactamase. The modified cephalosporin may or may not include the C-7 acylamino side chain. In various aspects, exclusion of the C-7 acylamino sidechain is beneficial for stabilizing the substrate in solution and reducing the intrinsic antibacterial properties of the substrate. The reaction of modified cephalosporin with a beta-lactamase to release a chromogenic agent (X) is shown below:

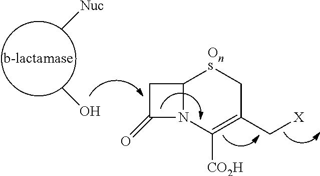

In some aspects, a detection agent includes a non-chromogenic substrate that reacts with a pathogenic component or product, and at least one chromogenic agent that reacts with a reaction product of the substrate and component or product to produce the visible indication (e.g., color appearance, color disappearance, color change). In certain aspects, the chromogenic agent interacts only with the reaction product and not with the unreacted substrate or pathogenic component or product. Accordingly, the visible indication is produced only when the chromogenic agent is combined with substrate that has been exposed to pathogen, and no visible indication is produced when the substrate and chromogenic agent are combined in the absence of pathogen. In certain aspects, the substrate and chromogenic agent are provided in a single solution that is applied to the object to be tested, while in other aspects, the substrate and chromogenic agent are provided as separate solutions that are simultaneously or sequentially applied to the object.

In some aspects, the substrate is a compound that reacts with beta-lactamases produced by a pathogen, such as a beta-lactamase inhibitor. Examples of beta-lactamase inhibitors include but are not limited to sulbactam, tazobactam, clavulanic acid, or avibactam, or combinations or derivatives thereof. Optionally, the beta-lactamase inhibitor to be used is selected based on solution stability (e.g., stability in slightly alkaline solutions for 12-24 months) and ease of chemical modification. In certain aspects, the beta-lactamase inhibitor reacts with a beta-lactamase to form an inactivated lactamase-inhibitor complex that can interact with a chromogenic agent to produce a visible indication (e.g., color change). Examples of chromogenic agents suitable for use with the beta-lactamase inhibitors described herein include but are not limited to dyes, such as triphenylene class dyes (e.g., cationic triphenylene class dyes such as fuschin, malachite green, malachite green oxalate, thymol blue, crystal violet), azo class dyes (e.g., methyl yellow, methyl orange), indigo class dyes (e.g., indigo carmine, indigo), or combinations thereof.

In some aspects, the detection agent includes a penicillanic acid sulfone (sulbactam or tazobactam) that is used in conjunction with a chromogenic agent. In certain aspects, the detection agent includes a penicillanic acid sulfone-based beta-lactamase inhibitor having the structure of Formula (I):

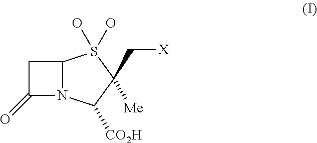

where X=H (sulbactam) or 1,2,3-triazole (tazobactam).

In certain aspects, penicillanic acid sulfones are advantageous in terms of solution stability (e.g., stable in a pH range from 4 to 8), amenability to chemistry modifications, and compatibility with chromogenic agents. In the presence of a pathogen that produces endogenous beta-lactamase(s) (e.g., penicillinase, cephalosporinase), the penicillanic acid sulfone (1) undergoes a multi-step hydrolysis to generate a hydrolyzed lactamase inhibitor conjugate (2) having a sulfinic acid moiety (—SO₂⁻). While the sulfinic acid moiety alone has no chromophore, it can form a covalent entity with a chromogenic agent (e.g., a hypervalent or cationic dye) to yield a hydrolyzed lactamase inhibitor-dye conjugate (3), which also results in a significant coloration change. The color change can be used as a visible indication to confirm the presence of certain pathogens. This reaction is shown below:

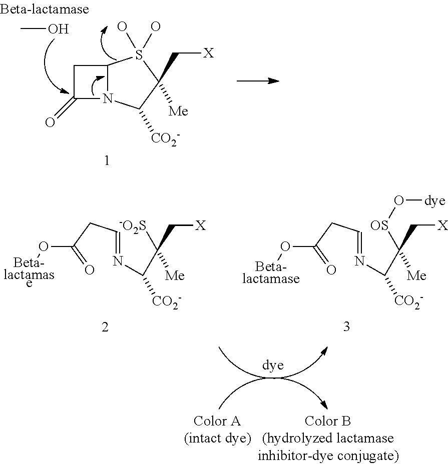

In some aspects, the detection agent includes a cephem acid sulfone that is used in conjunction with a chromogenic agent. In certain aspects, the detection agent includes a cephem acid sulfone-based beta-lactamase inhibitor having the structure of Formula (II):

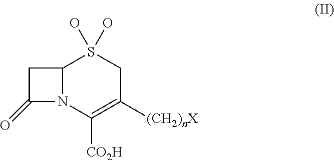

where n=0 or 1 and X=H, alkyl, akenyl, OAc or S-het. The detection methods described herein with respect to penicillanic acid sulfone-based beta-lactamase inhibitors are also applicable to cephem acid sulfone-based beta-lactamase inhibitors.

The detection agents of the present disclosure are suitable for use in detecting the presence or absence of pathogens on a wide variety of objects. In some aspects, the objects are located in and/or associated with settings or environments where pathogen contamination is particularly relevant, such as food preparation settings (e.g., kitchens, restaurants, dining areas, food processing and manufacturing facilities), healthcare settings (e.g., hospitals, clinics, nursing homes, inpatient facilities, outpatient facilities), laboratory settings (e.g., research facilities, biocontainment facilities), or combinations thereof. Examples of such objects include but are not limited to: food preparation devices and surfaces (e.g., countertops, cutting boards), food storage containers, medical devices and equipment (e.g., surgical equipment, hospital beds, surgical tables, surgical drapes, implantable devices, medications), research equipment and surfaces (e.g., benchtops, fume hoods, biosafety cabinets), protective equipment and uniforms (e.g., gloves, coats, gowns, scrubs, masks, face shields), or combinations thereof. In certain aspect, the object is a part of the body of a human or an animal, such as a body part that is relatively vulnerable to pathogen contamination and/or infection (e.g., skin, hands, mucous membranes, wound sites, or combinations thereof).

In certain aspects, the objects to be tested for pathogen contamination are adapted to be in contact with a human or animal body (e.g., medical devices and equipment, protective equipment and uniforms). In certain aspects, the objects are in contact to prepare items that will be in contact with a human or animal body (e.g., food preparation devices and surfaces). In certain aspects, the objects are body parts of a human or animal.

Accordingly, in order to minimize health risks and improve versatility of use, various aspects of the detection agents described herein are generally nontoxic (e.g., noncarcinogenic, nonteratogenic, non-irritating, non-corrosive, etc.) to humans, animals, and/or plants. In some aspects, the breakdown products of such detection agents are also generally nontoxic. In certain aspects, the detection agents described herein are generally environmentally acceptable, e.g., causing no harm to the environment, producing no toxic runoff, etc. In some aspects, the detection agents are designated by the American Food and Drug Administration (FD) as Generally Recognized As Safe (GRAS).

Moreover, in certain aspects, the detection agents of the present disclosure are generally non-staining in that they do not leave visible stains or residues on objects they come into contact with (e.g., garments, skin, surfaces). In certain aspects, the detection agents are easily removed from objects they come into contact with, e.g., by rinsing with a generally nontoxic solvent such as water. Alternatively or in addition, the detection agent includes a temporary visual indicator that provides a temporary visible indication, as previously described herein. In certain aspects, the detection agent includes a temporary visual indicator to provide the visible indication without leaving permanent stains or residues on the object.

The detection agents described herein are suitable for use in various formats. For example, in some aspects, the detection agent is provided in a solution that is applied to one or more portions of an object in order to test for the presence of pathogens on the object, e.g., by spraying, flowing, dripping, or wiping the solution onto the object portions and/or by immersing the object portions into the solution. In certain aspects, the solution includes a solvent that is generally nontoxic, non-staining, and/or environmentally acceptable. Examples of such solvents include but are not limited to water, ethanol, polyethylene glycol, propylene glycol, or combinations thereof. In various aspects, the solution is added to an absorbent material (e.g., a sponge, cloth, or membrane) and the absorbent material is applied to the object portions (e.g., by pressing or wiping) in order to bring the solution into contact with object portions.

As another example, in some aspects, the detection agent is embedded within a material, e.g., within the object, within a resin or other substance that is contacted with the object, or within a film, coating, or layer applied to a surface of the object. The material can be at least partially transparent or translucent, such that the visible indication generated by the detection agent is visually detectable from the exterior of the material.

In certain aspects, the detection agents provided herein are combined with each other in order to provide concurrent detection of multiple pathogen types. In certain aspects, two, three, four, five, six, seven, eight, nine, ten, or more different types of detection agents are combined (e.g., within a single solution or a single embedding material), with each type of detection agent adapted to detect different types of pathogens. In some aspects, the visible indications provided by each type of detection agent are visibly distinguishable from each other (e.g., having optical characteristics) so as to facilitate the determination of which pathogen types are present. Accordingly, in various aspects of the present disclosure, a kit is provided that includes multiple detection agents (e.g., provided separately or combined in a single delivery format) and a reference (e.g., a color scale or color key) that enables a user to interpret which pathogen types are associated with the various visible indications produced by the detection agents.

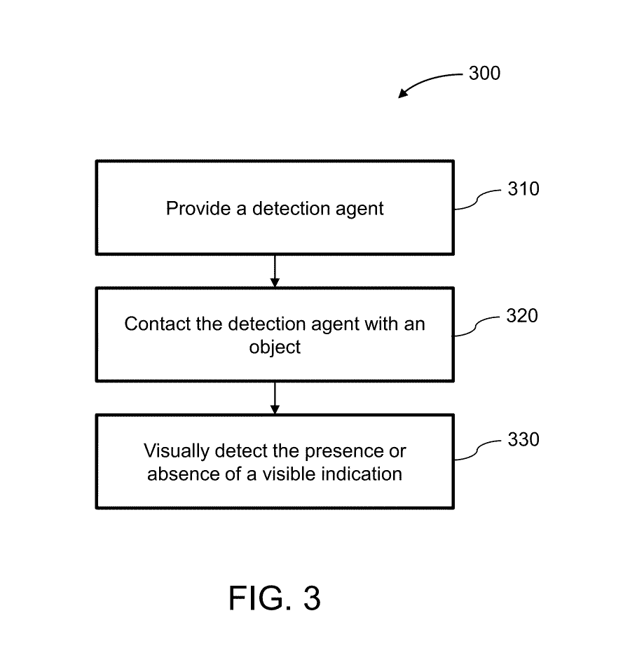

FIG. 3 illustrates a method 300 for detecting a pathogen on or within an object, in accordance with an aspect of the present disclosure. The method 300 is suitable for use with any aspect of the detection agents and objects described herein. In certain aspects, the method 300 is performed by a user (e.g., a healthcare professional, food preparation worker, researcher, inspector, etc.) in order to determine whether the object is contaminated with pathogens.

In step 310, a detection agent is provided. The detection agent is configured to generate a visible indication when exposed to a pathogen, as previously described herein. In some aspects, the detection agent is provided in a solution or embedded within a material, as discussed above. Optionally, the solution or material includes a plurality of detection agents each adapted to generate a different visible indication when exposed to different pathogen types.

In step 320, the detection agent is contacted with the object. In some aspects where the detection agent is integrally formed as part of the object (e.g., embedded within the object or within a film, coating or layer applied to the object), the step 320 occurs during manufacture of the object. In other aspects where the detection agent is provided separately from the object (e.g., as a solution or resin), the step 320 occurs after the object has already been manufactured.

In step 330, the presence or absence of the visible indication is detected, with the presence of the visible indication indicating that the pathogen is present on or within the object. As previously described herein, in various aspects, the detection of the visible indication is performed by a user with the unaided eye and without aid of visualization and/or detection devices. In some aspects, if the visible indication is detected, the user concludes that the object is contaminated with pathogen and takes appropriate measures, e.g., cleaning, sterilizing, additional testing, and/or disposal of the object.

In some aspects of the present disclosure, the detection agent is conjugated to one or more functional agents. In certain aspects, the detection agent includes one or more functional groups suitable for conjugation to a functional agent. Exemplary types of functional agents include but are not limited to: small molecules, peptides, proteins, lipids, sugars, nucleic acids, polymers, dendrimers, particles, beads, or combinations thereof. In certain aspects, the functional agent is a therapeutic used to treat a disease or condition. For instance, in certain aspects, the functional agent is an antibiotic used to treat pathogen infection, e.g., a beta-lactam antibiotic such as a penicillin, a cephalosporin, a monobactam, or a carbapenem. In some aspects, the antibiotic is used to treat the same pathogen detected by the detection agent, such that the detection agent can be applied to simultaneously detect and a treat a pathogen infection (e.g., at a wound site). In alternative aspects, the functional agent is an analog of an antibiotic, such as an analog exhibiting reduced or no antibacterial activity. In certain aspects, the antibiotic analog retains the ability to interact with (e.g., bind to) the pathogen, pathogen components, and/or pathogen product, but does not exhibit any killing and/or growth inhibitory activity.

Where a range of values is provided, it is understood that each intervening value, to the tenth of the unit of the lower limit unless the context clearly dictates otherwise, between the upper and lower limit of that range, and any other stated or intervening value in that stated range, is encompassed within the disclosure provided herein. The upper and lower limits of these smaller ranges can independently be included in the smaller ranges, and are also encompassed within the disclosure, subject to any specifically excluded limit in the stated range. Where the stated range includes one or both of the limits, ranges excluding either or both of those included limits are also included in the disclosure provided herein.

The specific dimensions of any of the apparatuses, devices, systems, and components thereof, of the present disclosure can be readily varied depending upon the intended application, as will be apparent to those of skill in the art in view of the disclosure herein. Moreover, it is understood that the examples and aspects described herein are for illustrative purposes only and that various modifications or changes in light thereof can be suggested to persons skilled in the art and are included within the spirit and purview of this application and scope of the appended claims. Numerous different combinations of aspects described herein are possible, and such combinations are considered part of the present disclosure. In addition, all features discussed in connection with any one aspect herein can be readily adapted for use in other aspects, herein. The use of different terms or reference numerals for similar features in different aspects does not necessarily imply differences other than those expressly set forth. Accordingly, the present disclosure is intended to be described solely by reference to the appended claims, and not limited to the aspects disclosed herein.

Unless otherwise specified, the presently described methods and processes can be performed in any order. For example, a method describing steps (a), (b), and (c) can be performed with step (a) first, followed by step (b), and then step (c). Or, the method can be performed in a different order such as, for example, with step (b) first followed by step (c) and then step (a). Furthermore, those steps can be performed simultaneously or separately unless otherwise specified with particularity.

While preferred aspects of the present disclosure have been shown and described herein, it is to be understood that the disclosure is not limited to the particular aspects of the disclosure described below, as variations of the particular aspects can be made and still fall within the scope of the appended claims. It is also to be understood that the terminology employed is for the purpose of describing particular aspects of the disclosure, and is not intended to be limiting. Instead, the scope of the present disclosure is established by the appended claims. In this specification and the appended claims, the singular forms “a,” “an” and “the” include plural reference unless the context clearly dictates otherwise.

All publications, patents, and patent applications mentioned in this specification are herein incorporated by reference to the same extent as if each individual publication, patent, or patent application was specifically and individually indicated to be incorporated by reference.

The specific dimensions of any of the apparatuses, devices, systems, and components thereof, of the present disclosure can be readily varied depending upon the intended application, as will be apparent to those of skill in the art in view of the disclosure herein. Moreover, it is understood that the examples and aspects described herein are for illustrative purposes only and that various modifications or changes in light thereof can be suggested to persons skilled in the art and are included within the spirit and purview of this application and scope of the appended claims. Numerous different combinations of aspects described herein are possible, and such combinations are considered part of the present disclosure. In addition, all features discussed in connection with any one aspect herein can be readily adapted for use in other aspects herein. The use of different terms or reference numerals for similar features in different aspects does not necessarily imply differences other than those expressly set forth. Accordingly, the present disclosure is intended to be described solely by reference to the appended claims, and not limited to the aspects disclosed herein.

### EXAMPLES

The following examples are included to further describe some aspects of the present disclosure, and should not be used to limit the scope of the invention.

### Example 1

### Detection of Pathogens Using a Malachite Green Derivative

This example describes the use of a malachite green derivative detection agent that exhibits rapid appearance and disappearance of color when exposed to a pathogen.

The malachite green dye is chemically modified to introduce metabolic breaking points, the addition of permeability enhancers, ionic strength adjustments, and/or addition of a metallic center in order to: (1) bind to bacterial cell walls; (2) exhibit rapid appearance and disappearance of color upon binding; and (3) eliminate runoff and toxicity from the dye or breakdown products.

Upon binding to the bacterial cell wall, the detection agent reaches equilibrium within 3 seconds and exhibits a highly visible color. The color disappears after 45 second due to oxidation and/or metabolic breakdown by the bacterium. The color is visible to the naked eye when bacterial concentrations reach at least 10³ colony forming units (CFUs).

The detection agent is provided as a solution or spray that can be applied to surfaces and wound sites in order to detect pathogen contamination.

### Example 2

### Detection of Pathogens Using a Rhodamine 6G Derivative

This example describes the use of a rhodamine 6G derivative detection agent that exhibits rapid appearance and disappearance of color when exposed to a pathogen.

The rhodamine 6G dye is chemically modified to introduce metabolic breaking points, the addition of permeability enhancers, ionic strength adjustments, and/or addition of a metallic center in order to: (1) bind to bacterial cell walls; (2) exhibit rapid appearance and disappearance of color upon binding; and (3) eliminate runoff and toxicity from the dye or breakdown products.

Upon binding to the bacterial cell wall, the detection agent reaches equilibrium within 3 seconds and exhibits a highly visible color. The color disappears after 45 second due to oxidation and/or metabolic breakdown by the bacterium. The color is visible to the naked eye when bacterial concentrations reach at least 10³ colony forming units (CFUs).

The detection agent is provided as a solution or spray that can be applied to surfaces and wound sites in order to detect pathogen contamination.

### Example 3

### Detection of Pathogens Using an Evans Blue Derivative

This example describes the use of an Evans blue derivative detection agent that exhibits rapid appearance and disappearance of color when exposed to a pathogen.

The Evans blue dye is chemically modified to introduce metabolic breaking points, the addition of permeability enhancers, ionic strength adjustments, and/or addition of a metallic center in order to: (1) bind to bacterial cell walls; (2) exhibit rapid appearance and disappearance of color upon binding; and (3) eliminate runoff and toxicity from the dye or breakdown products.

Upon binding to the bacterial cell wall, the detection agent reaches equilibrium within 3 seconds and exhibits a highly visible color. The color disappears after 45 second due to oxidation and/or metabolic breakdown by the bacterium. The color is visible to the naked eye when bacterial concentrations reach at least 10³ colony forming units (CFUs).

The detection agent is provided as a solution or spray that can be applied to surfaces and wound sites in order to detect pathogen contamination.

### Example 4

### Detection of Pathogens Using Beta-Lactamase Inhibitors and Cationic Dyes

This example describes the use of a beta-lactamase inhibitor-based detection agent to produce a color change when exposed to a beta-lactamase-secreting pathogen. A detection agent solution is prepared using by combining beta-lactamase inhibitor with a cationic dye. The beta-lactamase inhibitor forms a complex with secreted beta-lactamase, which covalently interacts with the dye to produce a color change. The color change can be used as a visible indication of pathogen exposure.

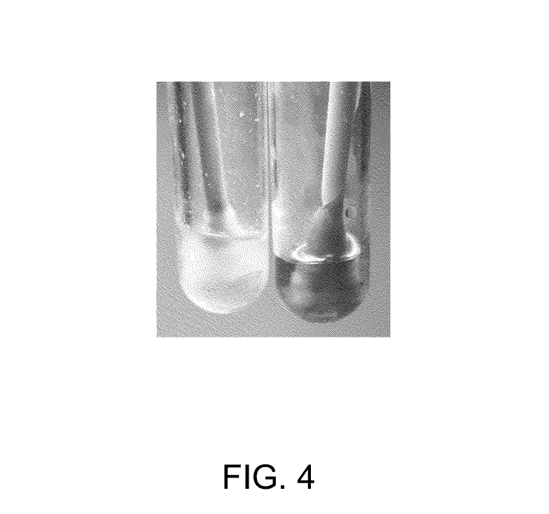

FIG. 4 illustrates detection of bacteria using a beta-lactamase inhibitor and methylene blue dye. A detection agent solution was prepared using a beta-lactamase inhibitor (tazobactam or sulbactam salt) and methylene blue. A blue to green color change was immediately observed upon addition of mixed Gram-positive and Gram-negative bacteria to the detection agent solution (FIG. 4, tube on left). Color disappearance was observed after a few hours, and may reflect trapping of bacteria and/or beta-lactamase by the formation of lactamase-inhibitor complexes. A control solution that was not exposed to bacteria did not exhibit any color change (FIG. 4, tube on right).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for detecting a pathogen on or within an object, the method comprising:
providing a detection agent configured to generate a visible indication when exposed to the pathogen;
contacting the detection agent with the object; and
visually detecting the presence or absence of the visible indication, wherein the presence of the visible indication indicates that the pathogen is present on or within the object.

2. The method of claim 1, wherein said pathogen is a bacterium, fungus, virus, parasite, prion, or a combination thereof.

3. The method of claim 2, wherein said pathogen is selected from Yersinia, Klebsiella, Providencia, Erwinia, Enterobacter, Salmonella, Serratia, Aerobacter, Escherichia, Pseudomonas, Shigella, Vibrio, Aeromonas, Streptococcus, Staphylococcus, Micrococcus, Moraxella, Bacillus, Clostridium, Corynebacterium, Francisella, Haemophilus, Bacteroides, Listeria, Acinetobacter, Brucella, Pasteurella, Flavobacterium, Actionmyces, Nocardia, Campylobacter, Mycobacterium, Candida, adenovirus, arenavirus, coronavirus, rhinovirus, influenza virus, picornavirus, paramyxovirus, reovirus, retrovirus, rhabdovirus, or a combination thereof.

4. The method of any one of claims 1-3, wherein said detection agent generates said visible indication when contacted with a plurality of different pathogen types.

5. The method of any one of claims 1-4, wherein said detection agent comprises a colorimetric agent, a fluorescent agent, a luminescent reagent, or a combination thereof.

6. The method of any one of claims 1-5, wherein said visible indication is detectable by an unaided eye.

7. The method of any one of claims 1-6, wherein said visible indication is generated by covalent or non-covalent binding of the detection agent with the pathogen.

8. The method of claim 7, wherein said visible indication is the emission of light of a first wavelength.

9. The method of claim 8, wherein said visible indication is absent when said detection agent is not bound to said pathogen.

10. The method of any one of claims 1 to 9, wherein said detection agent generates a second visible indication when said detection agent is not exposed to said pathogen.

11. The method of claim 10, wherein said second visible indication is the emission of light of a second wavelength.

12. The method of claim 11, wherein said first wavelength and said second wavelength are different wavelengths.

13. The method of claim 11, wherein said first wavelength and said second wavelength have different emission intensities.

14. The method of claim 13, wherein said emission intensity of the first wavelength is detectably different from said emission intensity of said second wavelength.

15. The method of claim 7, wherein said covalent binding is reversible.

16. The method of claim 7, wherein said binding to said pathogen is selective for a component located on said pathogen.

17. The method of claim 16, wherein said component is selected from a protein, peptide, lipid, lipopolysaccharide, oligosaccharide, polysaccharide, proteoglycan, glycoprotein, peptidoglycan, or a combination thereof.

18. The method of any one of claims 1 to 17, wherein said detection agent comprises a specific binding agent selected from DNA, RNA, oligonucleotides, polynucleotides, monoclonal antibodies, polyclonal antibodies, antibody fragments, single chain antibodies, synthetic antibodies, peptides, proteins, receptors, enzymes, DNA aptamers, RNA aptamers, oligosaccharides, lectins, peptoids, zDNA, peptide nucleic acids (PNAs), locked nucleic acids (LNAs), mimetics, small molecular weight compounds, or combinations thereof.

19. The method of claim 7, wherein said binding is non-specific binding.

20. The method of any one of claims 1-19, wherein said visible indication is generated by covalent or non-covalent binding of said detection agent with a component produced by said pathogen.

21. The method of claim 20, wherein said component comprises a protein, peptide, lipid, lipopolysaccharide, oligosaccharide, polysaccharide, proteoglycan, glycoprotein, peptidoglycan, or a combination thereof.

22. The method of claim 20, wherein said component comprises a metabolic byproduct of said pathogen.

23. The method of claim 22, wherein said metabolic byproduct is selected from carbon dioxide, ammonium, ammonia, hydrogen sulfide, sulfur dioxide, hydrogen, lactate, lactic acid, carbonic acid, sulfuric acid, or a combination thereof.

24. The method of claim 20, wherein said component comprises a gene product of said pathogen.

25. The method of claim 24, wherein said gene product is selected from beta-lactamase, LpxA, LpxB, LpxC, LpxD, or a combination thereof.

26. The method of claim 20, wherein said detection agent comprises a chromogenic substrate that generates a third visible indication upon binding with said component.

27. The method of claim 26, wherein said component oxidizes or reduces said chromogenic substrate.

28. The method of claim 26, wherein said component is an enzyme and said chromogenic substrate is a substrate for said enzyme.

29. The method of claim 28, wherein said chromogenic substrate is a chromogenic cephalosporin.

30. The method of any one of claims 1-29, wherein said detection agent is provided in a solution, and wherein said contacting the detection agent with the object comprises applying the solution to one or more portions of the object.

31. The method of claim 30, wherein said solution further comprises an additional detection agent configured to provide a fourth visible indication when exposed to a second pathogen.

32. The method of any one of claims 30-31, wherein said solution comprises an environmentally acceptable solvent.

33. The method of claim 32, wherein said environmentally acceptable solvent is water, ethanol, polyethylene glycol, propylene glycol or a combination thereof.

34. The method of any one of claims 30 to 33, wherein said applying the solution to said one or more portions of said object comprises spraying, flowing, dripping, or wiping the solution onto said one or more portions.

35. The method of any one of claims 30 to 33, wherein said applying the solution to said one or more portions of said object comprises immersing said one or more portions into said solution.

36. The method of any one of claims 1-35, wherein said detection agent is embedded within said object.

37. The method of any one of claims 1 to 36, wherein any one or more of said visible indication, said second visible indication, said third visible indication and said fourth visible indication is visually detectable from said exterior of the object.

38. The method of any one of claims 1 to 37, wherein said object comprises a resin, and wherein said detection agent is embedded in said resin.

39. The method of any one of claims 1-29, wherein said detection agent is associated with a film, coating, or layer located on a surface of said object.

40. The method of claim 39, wherein said detection agent is embedded within said film, coating, or layer located on said surface of said object.

41. The method of any one of claims 1-40, wherein said detection agent is generally non-toxic.

42. The method of any one of claims 1-41, wherein said detection agent is generally non-staining.

43. The method of any one of claims 1-42, wherein said object is located in a food preparation setting, a healthcare setting, or a laboratory setting.

44. The method of any one of claims 1-43, wherein any one or more of said visible indication, said second visible indication, said third visible indication, and said forth visible indication is a temporary visible indication.

45. The method of any one of claims 1-44, wherein said detection agent is selected from alizarin, alizarin yellow, basonyl green, basonyl blue, bromochlorophenol blue, bromoxylenol blue, bromophenol blue, bromopyrogallol red, bromothymol blue, bromocresol green, bromocresol purple, chlorophenol red, congo red, o-cresolphthalein, m-cresol purple, cresol red, m-cresol red, crystal violet, erichrome blue black R, erthyrosine powder, ethyl orange, Evans blue, fast sulphon black F, FD&C #2 indigotene, FD&C #2 lake, FD&C #1 triphenylmethane, FD&C #1 lake, FD&C #5 yellow, pyrazoine, FD&C #3 green, FD&C #3 red, FD&C #5 yellow lake, hydroxy naphthol blue, litmus powder, malachite green, malachite green oxalate, methyl orange, methyl red, methyl yellow, methylthymol blue, methyl violet, murexide powder, p-naphtholbenzein, neutral red, nitrazine yellow, pentamethoxy red, phenol red, phenophthalein, rhodamine 6 G, thymol blue, thymophthalein, triarylmethane (pylam blue), xylenol blue, or a combination thereof.

46. The method of claim 44, wherein said temporary visual indication is unstable when exposed to light.

47. The method of any one of claims 1-46, wherein said detection agent is conjugated to an antibiotic or a non-antibacterial analog thereof.

48. The method of claim 47, wherein the antibiotic is a beta-lactam antibiotic or a non-antibacterial analog thereof.

49. The method of any one of claims 1-44, wherein said detection agent comprises at least one chromogenic agent and a substrate, wherein said substrate reacts with a component produced by the pathogen thereby generating a reaction product, and said reaction product reacts with said chromogenic agent to generate a fifth visible indication.

50. The method of claim 49, wherein said substrate comprises a beta-lactamase inhibitor and said component is a beta-lactamase.

51. The method of claim 50, wherein said beta-lactamase inhibitor comprises a penicillanic acid sulfone or a cephem acid sulfone.

52. The method of any one of claims 49-51, wherein said chromogenic agent comprises a dye.

53. The method of claim 52, wherein said dye comprises a cationic triphenylene class dye, an azo class dye, an indigo class dye, or a combination thereof.

54. The method of claim 53, wherein said dye comprises a cationic triphenylene class dye comprising fuschin, malachite green, malachite green oxalate, thymol blue, crystal violet, or a combination thereof.

55. The method of claim 53, wherein said dye comprises an azo class dye selected from methyl yellow, methyl orange, and combinations thereof.

56. The method of claim 53, wherein said dye comprises an indigo class dye selected from indigo carmine, indigo, and combinations thereof.

57. The method of any one of claims 49-56, wherein said fifth visible indication comprises a color change in said chromogenic agent.
